(19) United States
(12) Patent Application Publication
(10) Pub. No.: US 2010/0284992 A1
(43) Pub. Date: Nov. 11, 2010

(54) Phospholipase C and Method of Use

(75) Inventors: Michael L. Vasil, Centennial, CO (US); Martin Stonehouse, Aurora, CO (US); Marsha A. Moses, Brookline, MA (US); Cecilia A. Fernandez, Jamaica Plain, MA (US); Adriana Vasil, Centennial, CO (US)

Correspondence Address: Don D. Cha, 547 Buena Vista Road, Golden, CO, 80401, US

(73) Assignee: The Regents of The University of Colorado, A Body Corporate, Denver, CO (US)

(21) Appl. No.: 12/443,957

(22) PCT Filed: Oct. 1, 2007
(86) PCT No.: PCT/US07/80044

§ 371 Date: May 13, 2010

Related U.S. Application Data
(60) Provisional application No. 60/827,836, filed on Oct. 2, 2006.

(51) Int. Cl. A61K 38/46; A61P 35/00; A61P 19/02; A61P 31/00; A61P 9/10

(52) U.S. Cl. 424/94.6

(57) ABSTRACT

The present invention provides a method for reducing angiogenesis using a phospholipase C.

### STATEMENT REGARDING FEDERALLY SPONSORED RESEARCH

The U.S. Government has a paid-up license in this invention and the right in limited circumstances to require the patent owner to license others on reasonable terms as provided for by the terms of Grant No. HL062608 awarded by the National Institutes of Health.

### FIELD OF THE INVENTION

The invention relates to a phospholipase C and methods for using the same.

### BACKGROUND OF THE INVENTION

Angiogenesis and vasculogenesis are processes involved in the growth of blood vessels. Angiogenesis is the process by which new blood vessels are formed from extant capillaries, while vasculogenesis involves the growth of vessels deriving from endothelial progenitor cells. Angiogenesis is a complex, combinatorial process that is regulated by a balance between pro- and anti-angiogenic molecules. Angiogenic stimuli (e.g., hypoxia or inflammatory cytokines) result in the induced expression and release of angiogenic growth factors such as vascular endothelial growth factor (VEGF) or fibroblast growth factor (FGF). These growth factors stimulate endothelial cells (EC) in the existing vasculature to proliferate and migrate through the tissue to form new endothelialized channels.

Angiogenesis and vasculogenesis, and the factors that regulate these processes, are important in embryonic development, inflammation, and wound healing, and also contribute to pathologic conditions such as tumor growth, diabetic retinopathy, rheumatoid arthritis, and chronic inflammatory diseases.

Both angiogenesis and vasculogenesis involve the proliferation of endothelial cells. Endothelial cells line the walls of blood vessels; capillaries are comprised almost entirely of endothelial cells. The angiogenic process involves not only increased endothelial cell proliferation, but also comprises a cascade of additional events, including protease secretion by endothelial cells, degradation of the basement membrane, migration through the surrounding matrix, proliferation, alignment, differentiation into tube-like structures, and synthesis of a new basement membrane. Vasculogenesis involves recruitment and differentiation of mesenchymal cells into angioblasts, which then differentiate into endothelial cells which then form de novo vessels.

Inappropriate, or pathological, angiogenesis is involved in the growth of atherosclerotic plaque, diabetic retinopathy, degenerative maculopathy, retrolental fibroplasia, idiopathic pulmonary fibrosis, acute adult respiratory distress syndrome, and asthma. Furthermore, tumor progression is associated with neovascularization, which provides a mechanism by which nutrients are delivered to the progressively growing tumor tissue.

The growth of blood vessels (a process known as angiogenesis) is essential for organ growth and repair. An imbalance in this process contributes to numerous malignant, inflammatory, ischemic, infectious and immune disorders.

While some angiogenesis inhibitors have recently been approved for treatment of a particular cancer, there is a continuing need for angiogenesis inhibitors.

### SUMMARY OF THE INVENTION

One aspect of the invention provides methods of reducing angiogenesis in an individual. The methods generally involve administering to the individual an effective amount of a phospholipase C. The methods are useful in treating conditions associated with or resulting from angiogenesis, such as pathological angiogenesis. The invention further provides methods of treating a condition associated with or resulting from angiogenesis.

Another aspect of the invention provides a method of reducing angiogenesis in a mammal. The method generally involves administering to a mammal a phosholipase C in an amount effective to reduce angiogenesis.

Still another aspect of the invention provides a method of treating a disorder associated with pathological angiogenesis. In some embodiments, the invention provides a method of inhibiting abnormal fibrovascular growth in a mammal. In some of these embodiments, the abnormal fibrovascular growth is associated with inflammatory arthritis. In some embodiments, the invention features a method of inhibiting a proliferative retinopathy in a mammal. In some of these embodiments, the proliferative retinopathy occurs as a result of diabetes in the mammal. The methods generally involve administering to a mammal a phospholipase C in an amount effective to reduce pathological angiogenesis.

Yet another aspect of the invention provides a method for inhibiting tumor growth in a mammal. In some embodiments, the invention provides a method of inhibiting pathological neovascularization associated with a tumor. The methods generally involve administering to a mammal a phospholipase C in an amount effective to reduce angiogenesis associated with a tumor. In some embodiments, the invention further comprises administering an anti-tumor chemotherapeutic agent other than a phospholipase C.

Suitable phospholipase C for use in the methods of the invention include, but are not limited to, PlcHR₂ (typically Pseudomonas aeruginosa PlcHR₂), Clostridium perfringens α-toxin, and a mixture thereof. The phospholipase C can be administered by any route of administration, including, but not limited to, intravenous, in or around a solid tumor, systemic, intraarterial, and topical.

One particular aspect of the invention provides a method for treating a disease or condition associated with angiogenesis in a subject, said method comprising administering a phospholipase C to the subject such that the phospholipase C reduces angiogenesis activity in said subject.

In some embodiments, the phospholipase C binds to an integrin receptor.

In other embodiments, the disease or condition associated with angiogenesis is cancer, macular degeneration, arthritis, or infectious diseases.

Still in other embodiments, the phospholipase C is a bacterial extracellular phospholipase C.

Yet in other embodiments, the phospholipase C is selected from the group consisting of PlcHR₂, Clostridium perfringens α-toxin, and a mixture thereof.

Another particular aspect of the invention provides a method for reducing or inhibiting angiogenesis in a subject comprising administering a phospholipase C to the subject.

Yet another aspect of the invention provides a method for selectively reducing proliferation of a cell comprising an integrin receptor, said method comprising contacting the cell with a phospholipase C such that the phospholipase C bind to the integrin receptor of the cell and reduces angiogenesis activity of the cell thereby reducing cell proliferation.

In some embodiments, the phospholipase C is a bacterial extracellular phospholipase C.

Still in other embodiments, the bacterial extracellular phospholipase C is selected from the group consisting of PlcHR₂, Clostridium perfringens α-toxin, and a mixture thereof.

Yet another particular aspect of the invention provides a method of reducing pathological angiogenesis in a mammal comprising administering to a mammal a phospholipase C in an amount effective to reduce pathological angiogenesis.

In some embodiments, the phospholipase C is a bacterial extracellular phospholipase C.

Still in other embodiments, the phospholipase C is selected from the group consisting of PlcHR₂, Clostridium perfringens α-toxin, and a mixture thereof.

Yet in other embodiments, the phospholipase C is administered by a route selected from the group consisting of intravenous, in or around a solid tumor, systemic, intraarterial, and topical.

Another particular aspect of the invention provides a method of inhibiting tumor growth in a mammal comprising administering to a mammal having a tumor a phospholipase in an amount effective to reduce angiogenesis thereby inhibiting tumor growth.

In some embodiments, the method further comprises administering an anti-tumor chemotherapeutic agent.

Still another particular aspect of the invention provides a method for inhibiting abnormal fibrovascular growth in a mammal comprising administering to a mammal having abnormal fibrovascular growth a phospholipase C in an amount effective to inhibit abnormal fibrovascular growth in the mammal.

In some embodiments, the phospholipase C binds to an integrin receptor thereby inhibiting abnormal fibrovascular growth in the mammal.

Yet in other embodiments, the abnormal fibrovascular growth is associated with inflammatory arthritis.

Another particular aspect of the invention provides a method of inhibiting a proliferative retinopathy in a mammal comprising administering to a mammal having proliferative retinopathy a phospholipase C in an amount effective to reduce the proliferative retinopathy in the mammal.

Yet in some other embodiments, the proliferative retinopathy occurs as a result of diabetes or aging in the mammal.

Still another aspect of the invention provides a method of inhibiting pathological neovascularization associated with a tumor in a mammal comprising administering to a mammal having a tumor a phospholipase C in an amount effective to inhibit the tumor-associated pathological neovascularization.

### BRIEF DESCRIPTION OF THE DRAWINGS

FIG. 1 is a schematic illustration of BD BioCoat™ Endothelial Cell Invasion and Migration Angiogenesis System;

FIG. 2 is a bar graph showing sensitivity of HUVECs and CHOs to PlcHR₂ treatment;

FIG. 3 is a bar graph showing sensitivity of HeLa and L929 fibroblasts to PlcHR₂ treatment;

FIG. 4 is another graph showing sensitivity of HUVECs, CHO, HeLa, and L929 fibroblasts to PlcHR₂ treatment;

FIG. 5 is a bar graph showing that PlcHR₂ activates caspase-3 in HUVEC;

FIG. 6 is a bar graph showing that the pan-caspase inhibitor Z-VAD-FMK inhibits PlcHR₂ activation of caspase-3;

FIG. 7 is a bar graph showing PlcHR₂ and the Clostridium perfringens α-toxin inhibit endothelial cell migration;

FIG. 8 is a bar graph showing the result of endothelial cell invasion inhibition assay of PlcHR₂ and heat-inactivated PlcHR₂ (i.e., ΔPlcHR₂);

FIG. 9 is 20X view of endothelial tube formation on matrigel at various PlcHR₂ concentration;

FIG. 10 is 20X view of endothelial tube break down at various PlcHR₂ concentrations; and

FIG. 11 is a picture of chick CAM assay showing PlcHR₂ suppresses embryonic angiogenesis.

### DETAILED DESCRIPTION OF THE INVENTION

### Definitions

The terms “treatment”, “treating” and the like are used herein to generally mean obtaining a desired pharmacologic and/or physiologic effect, e.g., reduction of angiogenesis and/or vasculogenesis. The effect can be prophylactic in terms of completely or partially preventing a disease or symptom thereof and/or can be therapeutic in terms of a partial or complete cure for a disease and/or adverse effect attributable to the disease due to angiogenesis. “Treatment” as used herein covers any treatment of a disease in a mammal, particularly a human, and includes: (a) preventing a disease or condition from occurring in a subject who may be predisposed to the disease but has not yet been diagnosed as having it; (b) inhibiting the disease, e.g., arresting its development; or (c) relieving the disease or its symptom. Reduction of angiogenesis and/or vasculogenesis is employed for subject having a disease or condition amenable to treatment by reducing angiogenesis.

The term “reduction” in reference to treating a disease means to suppress, reduce, or inhibit progression or development of the disease.

By “therapeutically effective amount it is meant an amount effective to facilitate a desired therapeutic effect, e.g., a desired reduction of angiogenesis and/or vasculogenesis. The desired therapeutic effect will vary according to the condition to be treated.

It must be noted that as used herein and in the appended claims, the singular forms “a”, “and”, and “the” include plural referents unless the context clearly dictates otherwise. Thus, for example, reference to “a phospholipase C” includes a plurality of phospholipase C's and reference to “the method” includes reference to one or more methods and equivalents thereof known to those skilled in the art, and so forth. Angiogenesis

Angiogenesis is the formation of new blood vessels from the pre-existing vasculature and is essential inter alia for growth, wound repair, and homeostasis. However, there are diseases that result in either excessive (e.g., vascular tumors and rheumatoid arthritis) or insufficient (e.g., macular degeneration and myocardial infarction) blood vessel formation. Angiogenesis is also involved in the progression of small, localized neoplasms to larger, growing, and potentially metastatic tumors. The principle cells involved in angiogenesis are endothelial cells, which line blood vessels. In the process of angiogenesis, endothelial cells go through a series of steps, including activation, basement membrane degradation, migration, extracellular matrix invasion, proliferation, and vessel formation.

In the embryo, blood vessels provide the growing organs with the necessary oxygen to develop. Apart from their nutritive function, vessels also provide instructive trophic signals to promote organ morphogenesis. Blood vessels arise from endothelial precursors, which share an origin with haematopoietic progenitors. This close link between the blood and blood vascular systems remains important for angiogenesis throughout life, even in disease. These progenitors assemble into a primitive vascular labyrinth of small capillaries—a process known as vasculogenesis. Interestingly, already at this stage capillaries have acquired an arterial and venous cell fate, indicating that vascular-cell specification is genetically programmed and not only determined by haemodynamic force. During the angiogenesis phase, the vascular plexus progressively expands by means of vessel sprouting and remodels into a highly organized and stereotyped vascular network of larger vessels ramifying into smaller ones. Nascent endothelial-cell (EC) channels become covered by pericytes (PCs) and smooth muscle cells (SMCs), which provide strength and allow regulation of vessel perfusion, a process termed arteriogenesis. The lymphatic system develops differently, as most lymphatics transdifferentiate from veins. Genetic studies in mice, zebrafish and tadpoles have provided insights into the key mechanisms and molecular players that regulate the growth of blood vessels (angiogenesis) or lymph vessels (lymphangiogenesis) in the embryo. For instance, members of the Notch family drive the arterial gene programme, whereas the orphan receptor COUP-TFII regulates venous specification. The homeobox gene Prox-1, by contrast, is a master switch of lymphatic commitment. VEGF and its homologue VEGF-C are key regulators of vascular and lymphatic EC sprouting, respectively, whereas platelet-derived growth factor (PDGF)-BB and angiopoietin-1 recruit mural cells around endothelial channels. The formation of vessels is a complex process, requiring a finely tuned balance between numerous stimulatory and inhibitory signals, such as integrins, angiopoietins, chemokines, junctional molecules, oxygen sensors, endogenous inhibitors and many others. An exciting recent development is the discovery of the links between vessels and nerves and, in particular, how axon-guidance signals such as Ephrins, Semaphorins, Netrins and Slits allow vessels to navigate to their targets or control vessel morphogenesis.

Angiogenic signals also guide axons and affect neurons in health and disease. Vessels of disease and death after birth, angiogenesis still contributes to organ growth but, during adulthood, most blood vessels remain quiescent and angiogenesis occurs typically only in the cycling ovary and in the placenta during pregnancy. However, ECs retain their remarkable ability of dividing rapidly in response to a physiological stimulus, such as hypoxia for blood vessels and inflammation for lymph vessels. As such, (lymph)angiogenesis is reactivated during wound healing and repair. But in many disorders, this stimulus becomes excessive, and the balance between stimulators and inhibitors is tilted, resulting in a (lymph)angiogenic switch. The best-known conditions in which angiogenesis is switched on are malignant, ocular and inflammatory disorders, but many additional processes are affected, such as obesity, asthma, diabetes, cirrhosis, multiple sclerosis, endometriosis, AIDS, bacterial infections and autoimmune disease, etc. There is even a close link between angiogenesis, neural stem cells and learning.

In other diseases, such as ischaemic heart disease or preeclampsia, the angiogenic switch is insufficient, causing EC dysfunction, vessel malformation or regression, or preventing revascularization, healing and regeneration. Besides its vascular activity, VEGF is also trophic for nerve cells, lung epithelial cells and cardiac muscle fibres, further explaining why insufficient VEGF levels contribute to neurodegeneration, respiratory distress and, possibly, cardiac failure. Angiogenesis has been implicated in more than 70 disorders so far, and the list is growing.

Vascular disease and septicemia are commonly observed during P. aeruginosa infections of immunocompromised patients. The pathogenesis of disseminated infections depends on the interaction of P. aeruginosa with blood vessels. To transverse the endothelial barrier and invade deeper tissues, the bacteria have to adhere to and damage endothelial cells. It has been demonstrated that P. aeruginosa can establish a nidus of infection immediately peripheral to the endothelial cells lining the vasculature where it can penetrate the endothelial lining of the vessels and either seed to the blood stream or invade into tissues. Infected foci are often complicated by vasculitis and thrombosis and serve as sites for the replication and seeding of the blood with bacteria. Furthermore, in vitro studies have shown that P. aeruginosa is able to invade and destroy endothelial cells, therefore suggesting that P. aeruginosa may produce products that could potentially be used as anti-angiogenic drugs.

P. aeruginosa produces numerous virulence factors, which include structural components, toxins, and various enzymes that contribute to its success as an opportunistic pathogen. Some of the virulence factors include exotoxin A, LasA, LasB, exoenzyme S, exoenzyme T, and an assortment of phospholipases (PlcH, PlcN, PlcB, and PlcA). The sensitivity of endothelial cells to the P. aeruginosa hemolytic phospholipase C (PlcH) (FIGS. 2,3 & 4) is believed to be relevant to the high mortality, blood borne infections caused by P. aeruginosa and suggest its potential use as an angiogenesis inhibitor.

PlcH was the first member of a now large family of enzymes, which have phosphatidylcholine specific phospholipase C (PC-PLC), sphingomyelinase (SMase), and phosphatase activity and are found in a number of microbial pathogens including Mycobacterium tuberculosis, Bordetella pertussis, Francisella tularensis, Burkholderia pseudomallei, and Xanthomonas campestris. PC-PLC hydrolyzes PC, yielding diacylglycerol (DAG) and phosphocholine, whereas SMases hydrolyze sphingomyelin yielding ceramide and phosphocholine. In mammalian systems, the products ceramide and DAG are believed to be involved in powerful signal transduction cascades. For example, ceramide has been shown to be involved in the eukaryotic stress response including regulation of growth, differentiation, and apoptosis, whereas DAG is believed to be involved in transformation, proliferation, and inflammation.

It is believed that most conventional angiogenesis inhibitors currently on the market are effective through their action on vascular endothelial growth factor, rather than acting directly on the endothelial cells. The present inventors have found that extracellular virulence factors of the opportunistic pathogen Pseudomonas aeruginosa and other extracellular bacterial proteins selectively and/or specifically inhibits and/or kills, at very low concentrations (as low as picomolar concentrations) human vascular endothelial cells. Surprisingly and unexpectedly, however, it has been found that these proteins, e.g., phospholipase C such as PlcHR₂ (typically Pseudomonas aeruginosa PlcHR₂), have a relatively very low toxicity to other types of cells (e.g., epithelial and fibrolasts). It has also been found that such proteins, e.g., PlcHR₂, require a specific integrin receptor for it to be toxic. It is believed that endothelial cells have this receptor and less susceptible cells do not. It is believed that binding to the integrin receptor in and of itself is not the primary reason for phospholipase C's angiogenesis activity. Without being bound to any theory, it is believed that PlcHR₂ first binds to an integrin receptor but to accomplish its anti-angiogenic activity it also needs to have phospholipase C activity. Regardless of its mode of action, phospholipase C's angiogenesis activity requires more than mere binding to an integrin receptor.

### Pharmaceutical Compositions

Upon reading the present specification, the ordinarily skilled artisan will appreciate that the pharmaceutical compositions comprising a phospholipase C described herein can be provided in a wide variety of formulations. For example, the phospholipase C can be formulated into pharmaceutical compositions by combination with appropriate, pharmaceutically acceptable carriers or diluents, and can be formulated into preparations in solid, semi-solid (e.g., gel), liquid or gaseous forms, such as tablets, capsules, powders, granules, ointments, solutions, suppositories, injections, inhalants and aerosols.

The phospholipase C formulation used will vary according to the condition or disease to be treated, the route of administration, the amount of phospholipase C to be administered, and other variables that will be readily appreciated by the ordinarily skilled artisan. In general, administration of phospholipase C can be either systemic or local, and can be achieved in various ways, including, but not necessarily limited to, administration by a route that is oral, parenteral, intravenous, intra-arterial, inter-pericardial, intramuscular, intraperitoneal, intra-articular, intra-ocular, topical, transdermal, transcutaneous, subdermal, intradermal, intrapulmonary, etc.

In pharmaceutical dosage forms, the phospholipase C can be administered in the form of their pharmaceutically acceptable salts, or they can also be used alone or in appropriate association, as well as in combination, with other pharmaceutically active compounds, such as an anti-tumor agent. The following methods and excipients are merely exemplary and are in no way limiting.

The phospholipase C can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

Formulations suitable for topical, transcutaneous, and transdermal administration can be similarly prepared through use of appropriate suspending agents, solubilizers, thickening agents, stabilizers, and preservatives. Topical formulations can be also utilized with a means to provide continuous administration of phospholipase C by, for example, incorporation into slow-release pellets or controlled-release patches.

The phospholipase C can also be formulated in a biocompatible gel, which gel can be applied topically or implanted (e.g., to provide for sustained release of phospholipase C at an internal treatment site). Suitable gels and methods for formulating a desired compound for delivery using the gel are well known in the art (see, e.g., U.S. Pat. Nos. 5,801,033; 5,827,937; 5,700,848; and MATRIGEL™).

For oral preparations, the phospholipase C can be used alone or in combination with appropriate additives to make tablets, powders, granules or capsules, for example, with conventional additives, such as lactose, mannitol, corn starch or potato starch; with binders, such as crystalline cellulose, cellulose derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The phospholipase C can be utilized in aerosol formulation to be administered via inhalation. The compounds of the invention can be formulated into pressurized acceptable propellants such as dichlorodifluoromethane, propane, nitrogen and the like.

Furthermore, the phospholipase C can be made into suppositories by mixing with a variety of bases such as emulsifying bases or water-soluble bases. The compounds of the invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions can be provided wherein each dosage unit, for example, teaspoonful, tablespoonful, tablet or suppository, contains a predetermined amount of the composition containing one or more inhibitors. Similarly, unit dosage forms for injection or intravenous administration can comprise the inhibitor(s) in a composition as a solution in sterile water, normal saline or another pharmaceutically acceptable carrier.

The term unit dosage form, as used herein, refers to physically discrete units suitable as unitary dosages for human and/or animal subjects, each unit containing a predetermined quantity of phospholipase C calculated in an amount sufficient to produce the desired reduction in angiogenesis in association with a pharmaceutically acceptable diluent, carrier or vehicle. The specifications for the unit dosage forms of the invention depend on the particular compound employed and the effect to be achieved, and the pharmacodynamics associated with each compound in the host.

The pharmaceutically acceptable excipients, such as vehicles, adjuvants, carriers or diluents, are readily available to the public. Moreover, pharmaceutically acceptable auxiliary substances, such as pH adjusting and buffering agents, tonicity adjusting agents, stabilizers, wetting agents and the like, are readily available to the public.

In some embodiments, a phospholipase C is administered in a combination therapy with one or more additional therapeutic agents. Exemplary therapeutic agents include therapeutic agents used to treat cancer, atherosclerosis, proliferative retinopathies, chronic arthritis, psoriasis, hemangiomas, etc.

### Dose

The dose of phospholipase C administered to a subject, particularly a human, in the context of the invention should be sufficient to effect a therapeutic reduction in angiogenesis in the subject over a reasonable time frame. The dose is determined by, among other considerations, the potency of the particular phospholipase C employed and the condition of the subject, as well as the body weight of the subject to be treated. For example, the level or affinity or both of the phospholipase C for the integrin receptor can play a role in regulating the compound's anti-angiogenic activity. The size of the dose also is determined by the existence, nature, and extent of any adverse side-effects that might accompany the administration of a particular compound.

In determining the effective amount of phospholipase C in the reduction of angiogenesis, the route of administration, the kinetics of the release system (e.g., pill, gel or other matrix), and the potency of the nicotine agonist are considered so as to achieve the desired anti-angiogenic effect with minimal adverse side effects. The phospholipase C is typically administered to the subject being treated for a time period ranging from a day to a few weeks, consistent with the clinical condition of the treated subject.

As will be readily apparent to the ordinarily skilled artisan, the dosage is adjusted for phospholipase C according to their potency and/or efficacy. A dose can be in the range of about 0.01 mg to 1000 mg, given 1 to 20 times daily, and can be up to a total daily dose of about 0.1 mg to 100 mg. If applied topically, for the purpose of a systemic effect, the patch or cream is designed to provide for systemic delivery of a dose in the range of about 0.01 mg to 1000 mg. If the purpose of the topical formulation (e.g., cream) is to provide a local anti-angiogenic effect, the dose would likely be in the range of about 0.001 mg to 1 mg. If injected for the purpose of a systemic effect, the matrix in which the phospholipase C is administered is designed to provide for a systemic delivery of a dose in the range of about 0.001 mg to 1 mg. If injected for the purpose of a local effect, the matrix is designed to release locally an amount of phospholipase C in the range of about 0.001 mg to 1 mg.

Regardless of the route of administration, the dose of phospholipase C can be administered over any appropriate time period, e.g., over the course of 1 to 24 hours, over one to several days, etc. Furthermore, multiple doses can be administered over a selected time period. A suitable dose can be administered in suitable subdoses per day, particularly in a prophylactic regimen. The precise treatment level is dependent upon the response of the subject being treated.

### Combination Therapy

In some embodiments, a phospholipase C is administered in a combination therapy with one or more other therapeutic agents, including an inhibitor of angiogenesis; and a cancer chemotherapeutic agent.

Suitable chemotherapeutic agents include, but are not limited to, the alkylating agents, e.g., Cisplatin, Cyclophosphamide, Altretamine; the DNA strand-breakage agents, such as Bleomycin; DNA topoisomerase II inhibitors, including intercalators, such as Amsacrine, Dactinomycin, Daunorubicin, Doxorubicin, Idarubicin, and Mitoxantrone; the nonintercalating topoisomerase II inhibitors such as, Etoposide and Teniposide; the DNA minor groove binder Plicamycin; alkylating agents, including nitrogen mustards such as Chlorambucil, Cyclophosphamide, Isofamide, Mechlorethamine, Melphalan, Uracil mustard; aziridines such as Thiotepa; methanesulfonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine; antimetabolites, including folate antagonists such as Methotrexate and trimetrexate; pyrimidine antagonists, such as Fluorouracil, Fluorodeoxyuridine, CB3717, Azacytidine, Cytarabine; Floxuridine purine antagonists including Mercaptopurine, 6-Thioguanine, Fludarabine, Pentostatin; sugar modified analogs include Cyctrabine, Fludarabine; ribonucleotide reductase inhibitors including hydroxyurea; Tubulin interactive agents including Vincristine Vinblastine, and Paclitaxel; adrenal corticosteroids such as Prednisone, Dexamethasone, Methylprednisolone, and Prodnisolone; hormonal blocking agents including estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone estrogens, conjugated estrogens and Ethinyl Estradiol and Diethylstilbesterol, Chlorotrianisene and Idenestrol; progestins such as Hydroxyprogesterone caproate, Medroxyprogesterone, and Megestrol; androgens such as testosterone, testosterone propionate; fluoxymesterone, methyltestosterone; and the like.

The phospholipase C can be administered with other anti-angiogenic agents. Anti-angiogenic agents include, but are not limited to, angiostatic steroids such as heparin derivatives and glucocorticosteroids; thrombospondin; cytokines such as IL-12; fumagillin and synthetic derivatives thereof, such as AGM 12470; interferon-cc; endostatin; soluble growth factor receptors; neutralizing monoclonal antibodies directed against growth factors; and the like.

Reducing Angiogenesis in vivo

The invention provides a method of reducing angiogenesis in a mammal. The method generally involves administering to a mammal a phospholipase C in an amount effective to reduce angiogenesis. An effective amount of a phospholipase C reduces angiogenesis by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, or more, when compared to an untreated (e.g., a placebo-treated) control.

Whether angiogenesis is reduced can be determined using any known method. Methods of determining an effect of an agent on angiogenesis are known in the art and include, but are not limited to, inhibition of neovascularization into implants impregnated with an angiogenic factor; inhibition of blood vessel growth in the cornea or anterior eye chamber; inhibition of endothelial cell proliferation, migration or tube formation in vitro; the chick chorioallantoic membrane assay; the hamster cheek pouch assay; the polyvinyl alcohol sponge disk assay. Such assays are well known in the art and have been described in numerous publications, including, e.g., Auerbach et al., Pharmac. Ther., 1991, 51,1-11, and references cited therein.

The invention also provides methods for treating a condition or disorder associated with or resulting from pathological angiogenesis. In the context of cancer therapy, a reduction in angiogenesis according to the methods of the invention effects a reduction in tumor size and/or a reduction in tumor metastasis. Whether a reduction in tumor size is achieved can be determined, e.g., by measuring the size of the tumor, using standard imaging techniques. Whether metastasis is reduced can be determined using any known method. Methods to assess the effect of an agent on tumor size are well known, and include imaging techniques such as computerized tomography and magnetic resonance imaging.

### Conditions Amenable to Treatment

Any condition or disorder that is associated with or that results from pathological angiogenesis, or that is facilitated by neovascularization (e.g., a tumor that is dependent upon neovascularization), is amenable to treatment with a phospholipase C.

Conditions and disorders amenable to treatment include, but are not limited to, cancer; atherosclerosis; proliferative retinopathies such as diabetic retinopathy, age-related maculopathy, retrolental fibroplasia; excessive fibrovascular proliferation as seen with chronic arthritis; psoriasis; and vascular malformations such as hemangiomas, and the like.

The instant methods are useful in the treatment of both primary and metastatic solid tumors, including carcinomas, sarcomas, leukemias, and lymphomas. Of particular interest is the treatment of tumors occurring at a site of angiogenesis. Thus, the methods are useful in the treatment of any neoplasm, including, but not limited to, carcinomas of breast, colon, rectum, lung, oropharynx, hypopharynx, esophagus, stomach, pancreas, liver, gallbladder and bile ducts, small intestine, urinary tract (including kidney, bladder and urothelium), female genital tract, (including cervix, uterus, and ovaries as well as choriocarcinoma and gestational trophoblastic disease), male genital tract (including prostate, seminal vesicles, testes and germ cell tumors), endocrine glands (including the thyroid, adrenal, and pituitary glands), and skin, as well as hemangiomas, melanomas, sarcomas (including those arising from bone and soft tissues as well as Kaposi's sarcoma) and tumors of the brain, nerves, eyes, and meninges (including astrocytomas, gliomas, glioblastomas, retinoblastomas, neuromas, neuroblastomas, Schwannomas, and meningiomas). The methods are also useful for treating solid tumors arising from hematopoietic malignancies such as leukemias (i.e., chloromas, plasmacytomas and the plaques and tumors of mycosis fungoides and cutaneous T-cell lymphoma/leukemia) as well as in the treatment of lymphomas (both Hodgkin's and non-Hodgkin's lymphomas). In addition, the instant methods are useful for reducing metastases from the tumors described above either when used alone or in combination with radiotherapy and/or other chemotherapeutic agents.

Other conditions and disorders amenable to treatment using the methods of the instant invention include autoimmune diseases such as rheumatoid, immune and degenerative arthritis; various ocular diseases such as diabetic retinopathy, retinopathy of prematurity, corneal graft rejection, retrolental fibroplasia, neovascular glaucoma, rubeosis, retinal neovascularization due to macular degeneration, hypoxia, angiogenesis in the eye associated with infection or surgical intervention, and other abnormal neovascularization conditions of the eye; skin diseases such as psoriasis; blood vessel diseases such as hemangiomas, and capillary proliferation within atherosclerotic plaques; Osler-Webber Syndrome; plaque neovascularization; telangiectasia; hemophiliac joints; angiofibroma; and excessive wound granulation (keloids).

Additional objects, advantages, and novel features of this invention will become apparent to those skilled in the art upon examination of the following examples thereof, which are not intended to be limiting.

### EXAMPLES

Purification of the P. aeruginosa Hemolytic Phospholipase C, PlcHR₂

PlcHR₂ was purified using a batch purification process. Briefly, the preswollen microgranular anion exchanger diethylaminoethyl cellulose DE52 Sephacel (Whatman, Florham Park, N.J.) was equilibrated in buffer A (50 mM Tris-HCl, pH 7.2, 50 mM NaCl), overnight at 4° C. A 50 ml Lauria broth (LB), 800 μg/ml carbenicillin starter culture was inoculated with P. aeruginosa ADD1976::pADD1976 containing the plcHR_{1,2} genes. After 12 hours of growth at 37° C., the entire 50 ml starter culture was added to 200 ml LB, 800 μg/ml carbenicillin and grown at 37° C. for an additional 3 hours. The bacteria were harvested by centrifugation, washed with 100 ml M9 minimal media, and used to inoculate three 750 ml cultures of M9 minimal media, 200 μg/ml carbenicillin to a starting A₅₉₀ of 0.5. The bacteria were allowed to adapt to the M9 media for 1 hour at 37° C. and then induced by the addition of Isopropylthio-b-galactopyronaoside (IPTG) (Research Products International, Mt. Prospect, Ill.) to a final concentration of 2 mM. During induction, the PC-PLC activity of the supernatants was monitored with the PC-PLC synthetic substrate ρ-nitrophenyl-phosphorylcholine (NPPC) (Sigma, St. Louis, Mo.) as previously described (Stonehouse, M. J., et al., Mol Microbiol, 2002, 46(3), 661-76) and harvested when the activity peaked, usually after 2 hours. The ionic strength of the supernatant was reduced by the addition of 1.5 volumes (3375 ml) cold sterile ddH₂O and all 5625 ml were batch bound to 80 grams DE52 Sephacel (35 g/L supernatant) for 1 hour at 4° C. Following binding, the DE52 Sephacel was washed three times with 800 ml 4° C. buffer A and batch eluted with 300 ml 50 mM Tris-HCl (pH 7.2), 500 mM NaCl. The DE52 Sephacel eluate was concentrated via 75% ammonium sulfate precipitation, dialyzed extensively with buffer A, and loaded onto a 7.5% non-denaturing polyacrylamide preparative gel (Bio-Rad model 491, 27 mM diameter). Non-denaturing gel conditions for purification of PlcHR₂ included: upper chamber running buffer (40 mM Tris-HCl, pH 8.89, 40 mM glycine), lower chamber running buffer (60 mM Tris-HCl, pH 7.47), separating gel (237 mM Tris-HCl, pH 8.48), and stacking gel (40 mM Tris-HCl, pH 6.9). The preparative gel was run at 10 watts constant power for 16 hours, and proteins were eluted in 3 ml fractions using buffer A at a flow rate of 350 μl/min. All purification procedures were carried out at 4° C. unless otherwise noted. Fractions possessing PC-PLC activity were identified using NPPC and the fraction with peak activity was aliquoted and stored at −80° C.

### Cell Culture

Cell lines used in this study (see Table 1) were purchased from ATCC except for the human umbilical vein endothelial cells (HUVECs), which were purchased from BD Biosciences. Capillary endothelial cells were obtained from Children's Hospital in Boston and all growth media were purchased from ATCC and Gibco BRL. Cells were maintained via the manufactures' recommended procedures. F12K, Eagles's Minimum Essential Media and Dulbecco's Modified Eagle's Media were all supplemented with 10% fetal bovine serum. All cell were grown at 37° C. in 5% CO₂.

TABLE 1

| Cell Line | Organism | Morphology | Growth Media |
|---|---|---|---|
| HUVEC | Homo sapiens | endothelial | EGM ®-2 |
| CHO-K1 | Cricetulus | epithelial/ovary | F12K |
| | griseus | | |
| L929 | Mus musculus | fibroblast/areolar | Eagle's Minimum Essential Media |
| HeLa | Homo sapiens | epithelial/cervix | Eagle's Minimum Essential Media |
| J774 Macrophage | Mus musculus | Macrophage | Dulbecco's Modified Eagle's |
| | | | Medium (DMEM) |
| 1° Lung Epithelial | Homo sapiens | epithelial | Chemically Defined Medium |
| Capillary | Homo sapiens | Endothelial | DMEM with 3 ng/ml basic |
| Endothelial Cells | | | fibroblast growth factor (bFGF) |

### Lactate Dehydrogenase Cytotoxicity Assay

Various cell lines were challenged with PlcHR₂ to determine the cell specificity of PlcHR₂ using the CytoTox 96® Non-Radioactive Cytotoxicity assay (Promega, Madison, Wis.) following the manufactures' suggested protocol. The CytoTox 96® assay measures lactate dehydrogenase (LDH), a stable cytosolic enzyme that is released upon cell lysis or membrane damage. Released LDH in culture supernatants is measured with a coupled enzymatic assay that produces a red product that is detected spectrophotometrically at 490 nm. The intensity of the color formed is proportional to the amount of released LDH. Cell lines were cultivated in 24 or 96 well plates. When the cells reached 80 to 90% confluency, the spent media was exchanged with fresh media containing 2 ng/ml to 4.5 μg/ml of PlcHR₂. The cultures were incubated at 37° C., 5% CO₂ for 2 to 22 hours and absorbencies were read at 490 nm in a Bio-Rad Benchmark Microplate Reader. Percent LDH release was determined by subtraction of the blank from each reading and then dividing the resulting value by the total LDH release value.

### Caspase-3 Activation Assay

The aspartate-specific cysteinyl proteases or caspases are a set of mediators implicated in apoptosis. The activation of caspase-3 in mammalian cells is a hallmark of apoptosis. To assess whether the mode of death induced by PlcHR₂ was apoptotic or necrotic, caspase-3 activity was assayed with the colorimetric CaspACE Assay system (Promega, Madison, Wis.). The colorimetric substrate (Ac-DEVD-pNA) is hydrolyzed by activated caspase-3 releasing pNA producing a yellow color that is quantified at an absorbance of 405 nm. Camptothecin, a topoisomerase I inhibitor, was used as a positive control for activation of caspase-3 and induction of apoptosis. For inhibition of apoptosis Z-VAD-FMK was added to samples at a final concentration of 50 μM. HUVEC were cultivated in 6 well tissue culture dishes to 80-90% confluency at which time the media was exchanged with 2 ml of fresh media containing PlcHR₂ or other compounds to be examined. The cultures were allowed to incubate at 37° C. in 5% CO₂ for 3 to 16 hours. The cells were harvested by trypsin/EDTA treatment, washed with ice cold PBS and suspended in lysis buffer at a concentration of 1×10⁸ cells/ml. To prepare lysates cells were freeze-thawed four times and sonicated twice for 15 seconds at level 10 in a Sonic Dismembrator Model 100 (Fisher Scientific, Hampton, N.H.). The lysates were incubated on ice for 15 minutes before the cell lysate supernatant was harvested by centrifuge at 16,100×g for 20 minutes at 4° C. Caspase-3 activity in the cell lysates was assayed for by the manufactures' recommended protocol.

### Endothelial Cell Invasion and Migration

The effects of PlcHR₂ on endothelial cell invasion and migration, two important steps in the angiogenic process, were measured using the BD BioCoat™ Endothelial Cell Invasion and Migration Angiogenesis Systems (BD Biosciences, Bedford, Mass.) following the manufactures' recommended protocol. These angiogenesis systems are in vitro, quantitative assays that utilize a 24 multiwell BD Falcon™ FluoroBlok™ insert plate with a 3.0 micron pore size polyethylene terephthalate (PET) membrane that is uniformly coated with BD Matrigel™ matrix in the Invasion assay or Human Fibronectin in the Migration assay.

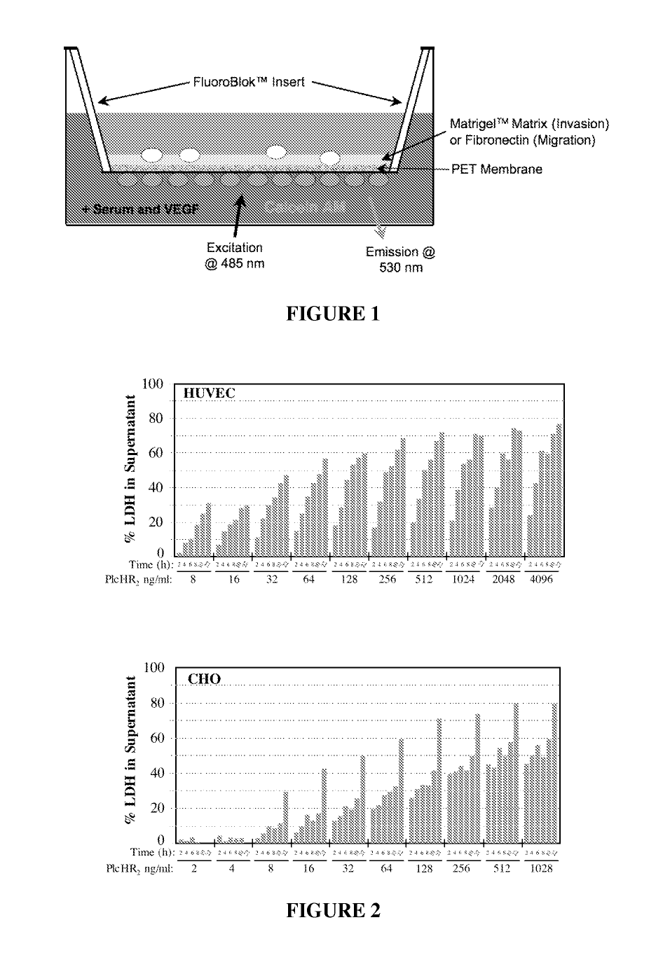

BD BioCoat™ Endothelial Cell Invasion and Migration Angiogenesis System is schematically illustrated in FIG. 1. Briefly Endothelial cell have to attach to the fibronectin (migration assay) or the BD Matrigel™ (invasion assay) and then move through the PET membrane towards the chemoattractant (Serum and VEGF). The cells are allowed to migrate, e.g., for 22 hours, and then they are stained with Calcein AM. Labeled cells (shown in green) that passed through the pores of the BD FluoroBlok™ insert are detected.

The BD Matrigel™ used in the invasion assay is a solubilized biologically active basement membrane preparation extracted from the Engelbreth-Holm-Swarm (EHS) mouse sarcoma cell line. See, for example, Kleinman, H. K., et al., Biochemistry, 1982, 21(24), 6188-93. BD Matrigel™ matrix provides a basement membrane that is a barrier to non-activated, non-invasive cells, but that allows the passage of activated, invasive endothelial cells moving towards an angiogenic stimulus. In the invasion assay the endothelial cells attach to, degrade and invade through the Matrigel™ while in the migration assay the cells attach to Human Fibronectin and then migrate to the bottom chamber (see FIG. 1).

HUVECs were grown in EGM-2 medium free of serum and vascular endothelial growth factor (VEGF) for 5 hours. The 24 multiwell BD Falcon™ FluoroBlok™ was hydrated by adding 500 μl of 37° C. Endothelial Cell Basal Medium-2 (EBM-2) (Clonetics, Walkersville, Md.) to the top insert wells and incubated for 30 minutes at 37° C. in 5% CO₂. Once hydrated, the basal media was removed and replaced with 200 μl of EGM-2 media (Clonetics, Walkersville, Md.) containing the test inhibitors but lacking serum and vascular endothelial growth factor (VEGF). Each respective bottom well received 750 μl of EGM-2 containing serum, VEGF and the same concentration of the test inhibitors. The serum and VEGF within the EGM-2 media act as the attractant for the endothelial cells to move to the bottom chamber (see FIG. 1). Fifty μl of a HUVEC suspension containing approximately 5.0×10⁴ cells were added to the upper insert chambers. Plates were incubated for 22 hours at 37° C. in 5% CO₂. Endothelial cell invasion and migration were measured by labeling the cells that passed through the FluoroBlok™ insert into the bottom chamber with 4 μg/ml Calcein AM (Molecular Probes, Eugene, Oreg.) for 90 minutes at 37° C. in 5% CO₂. The plate was then read in a fluorescent plate reader (Bio-TEK Synergy HT, Winooski, Vt.) at excitation and emission wavelengths of 485 and 530 nm, respectively.

### Capillary Endothelial Cell Proliferation

Once endothelial cells have invaded and migrated to new tissue they begin to proliferate. Therefore, to quantify inhibition of endothelial cell proliferation by PlcHR₂, the endothelial cell proliferation assay as described previously was used. See, for example, Moses, M. A., et al., Science, 1990, 248(4961), 1408-10; Moses, et al., J Cell Biol, 1992, 119(2), 475-82; Moses, M. A., et al., Proc Natl Acad Sci USA, 1999, 96(6), 2645-50; and O'Reilly, M. S., et al., Cell, 1994, 79(2), 315-28. Briefly, this assay typically works by detecting the phosphatases released from lysed cells via a colorimetric assay using the synthetic phosphatase substrate p-nitrophenyl-phosphate (NPP) (Sigma, St. louis, Mo.). A proliferating cell population releases more phosphatase upon lysis resulting in a greater colorimetric change.

Capillary endothelial cells, isolated from bovine adrenal cortex, were plated on pregelatinized 96-well plates at a density of 2000 cells per well in DMEM supplemented with 5% calf serum and allowed to attach for 24 hours. Cells were then treated with fresh media with (mitogen stimulated) or without 1 ng/ml bFGF and challenged with PlcHR₂. Control wells contained cells with medium alone or medium with bFGF. After 72 h, the media was removed, and the cells were lysed in buffer containing Triton X-100 and the phosphatase substrate NPP. After a 2-h incubation at 37° C., NaOH was added to each well to terminate the reaction and the cell density was determined by colorimetric analysis (absorbance at 410 nm) using a SpectraMax 190 multiwell plate reader (Molecular Devices, Sunnyvale, Calif.).

### Endothelial Tube Formation

To test the effects of PlcHR₂ on in vitro endothelial tube formation, endothelial cells on Matrigel™ were grown. Under these conditions endothelial cells are able to differentiate into capillary-like structures (Tubes) within 20 hours. One hundred and fifty μl of Matrigel™ was used to evenly coat each well of a 24 well plate. Following polymerization of the Matrigel™ for 30 minutes at 37° C., 5% CO₂, one ml of EGM-2 media containing PlcHR₂ was added to each well. Two hundred μl of a HUVEC cell suspension containing approximately 1×10⁵ cells in EGM-2 was then added to each well and plates were incubated for 24 hours at 37° C. in 5% CO₂. Tubes were visualized with a Nikon Eclipse TS 100 inverted microscope (Nikon, Japan) and pictures were taken with a Digital Sight DS-5M camera (Nikon, Japan) connected to a Digital Sight DS-L1 computer (Nikon, Japan).

### Chick Chorioallantoic Membrane (CAM) Assay

The chicken chorioallantoic membrane (CAM) assay was conducted as reported previously. See, for example, Moses, M. A., et al., Science, 1990, 248(4961), 1408-10; Moses, et al., J Cell Biol, 1992, 119(2), 475-82; Moses, M. A., et al., Proc Natl Acad Sci USA, 1999, 96(6), 2645-50; and O'Reilly, M. S., et al., Cell, 1994, 79(2), 315-28. Briefly, 3-day-old chick embryos were removed from their shells and incubated in Petri dishes for 3 days. On embryonic day 6, 5 ng/ml PlcHR₂ was mixed into methylcellulose discs and applied to the surfaces of developing CAMs, above the dense subectodermal plexus. After 48 h of incubation, the eggs were examined for vascular reactions under a dissecting scope (60X) and photographed.

### ρ-Nitrophenyl-Phosphorylcholine (NPPC) Enzymatic Assay

Enzymatic assays with the artificial substrate ρ-nitrophenyl-phosphorylcholine (NPPC) were carried as described by Kurioka et al., in Anal Biochem., 1976, 75(1), 281-289. It was determined that the suitable NPPC reaction conditions for PlcHR₂ consisted of 100 mM Tris-HCl (pH 7.2), 25% glycerol at 37° C. Beers law was used to convert the absorbance values to nmols of product for kinetic calculations. Beer's law states A=εcd, where A is the absorbance, ε is the extinction coefficient, c is the concentration, and d is the path length in cm. Based on the reaction conditions in a microtiter plate assay (ε for NPPC at 410 nM is 1.525×10⁴ mol⁻¹·L·cm⁻¹, d=0.25 cm for 100 μl) it was determine that an absorbance of 1 at 410 nM equated to 26.23 nmols of ρ-nitrophenol product produced. A typical assay consisted of taking A₄₁₀ readings every minute over a 5-minute period. These initial absorbencies were converted to nmols of ρ-nitrophenyl using the above conversion. Graphing the nmols of p-nitrophenyl vs. time provided the Vᵢₙₜ value in nmols ρ-nitophenyl·min⁻¹. These values were divided by the milligrams of PlcHR₂ to give the initial rates in nmols ρ-nitrophenol·min⁻¹·mg⁻¹ PlcHR₂. The initial rates of hydrolysis (μmol·min⁻¹·mg⁻¹) at different substrate concentrations were fitted to the Michaelis-Menten equation using the program Sigma Plot (SPSS inc.). The kinetic parameters Vₘₐₓ and Kₘ were obtained from the nonlinear least squares fit of the data; k_{cat} values were calculated using a Mᵣ of 96,000 Da for PlcHR₂. The kinetic parameters for PlcHR₂ in a NPPC assay were determined by assaying PlcHR₂ and the T178A PlcHR₂ mutant at 0.01 and 0.05 μg/ml, respectively with varying concentrations of NPPC (1.875, 3.75, 7.5, 15, 30, 60, 80, 100, 125, 150 mM).

### Random Mutagenesis of PlcH

PlcH was randomly mutagenized by using the inherent mutagenic rate of Taq polymerase. The plasmid template for mutagenesis was PstI linearized pUC18 containing the 3.12 kb PlcHR_{1,2} insert. The 5′ sense primer was PAMSf002 (AGGCACCCCAGGCTTTACAC) and the 3′ antisense primer was PAMSr001 (ATCCTTCCACGGCGGCACC) located 3′ of the XhoI restriction site. Two rounds of PCR were performed using standard PCR procedures. Following the first round of PCR the desired product was gel purified and then subjected to a second round of PCR with the same primers. Following the second PCR both the PCR product and pUC18 containing the 3.12 kb PlcHR_{1,2} insert were digested with BamHI and XhoI. The 1,118 by PCR fragment and the 4,656 by vector were gel purified, ligated together, and transformed into E. coli DH5α. The resulting transformants were then transferred to 96 well microtiter plates containing 100 μl LB with 100 μg/ml ampicillin and allowed to grow 12 h at 37° C. To store the mutant library 100 μl 50% sterile glycerol was added and the library frozen at −80 ° C. Clones deficient in activity on the synthetic substrate ρ-NPPC were sequenced and characterized.

### RESULTS

### Endothelial Cell Specificity and Cytotoxicity

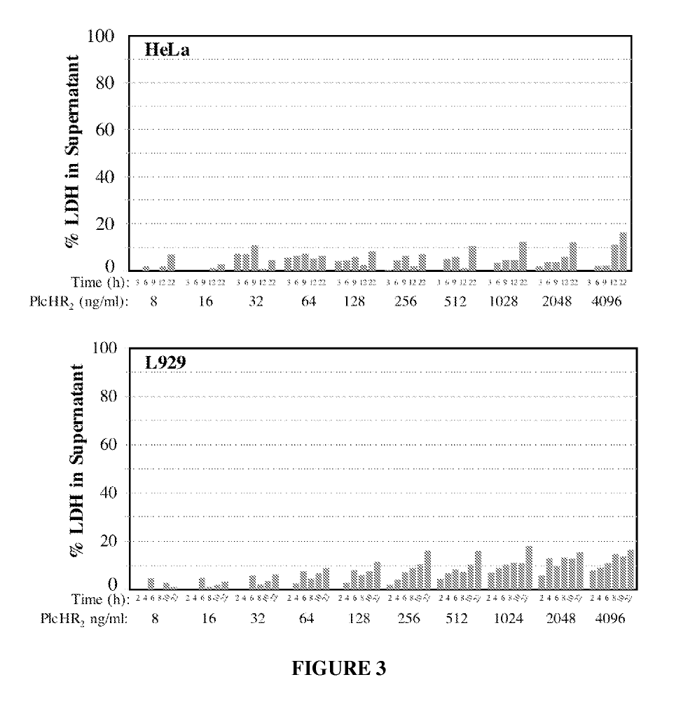

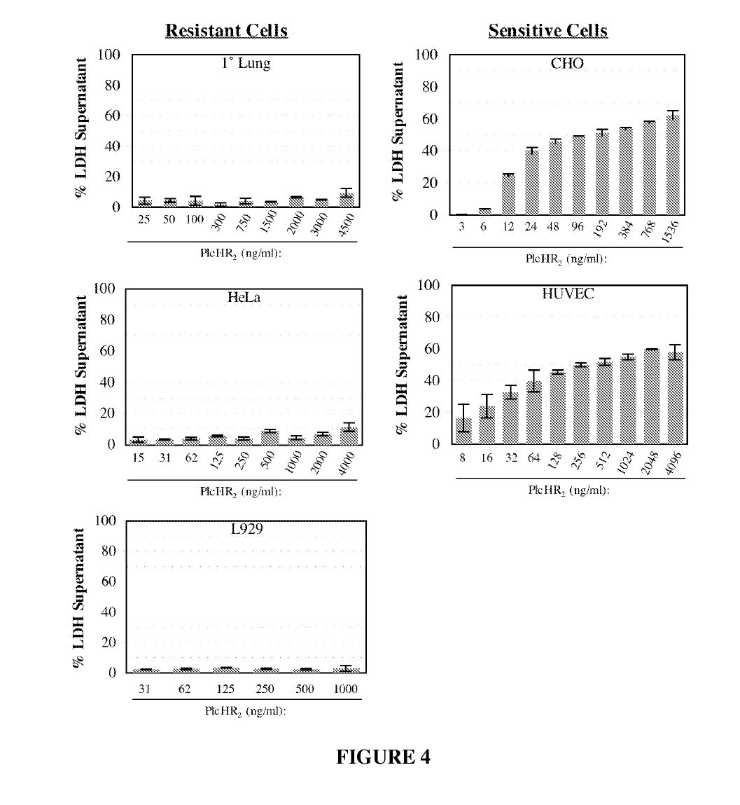

To examine the cell specificity of PlcHR₂, lactate dehydrogenase (LDH) release cytotoxicity assays were performed with a variety of cell lines listed in Table 1 above: HUVECs, HeLa, CHO cells, and L929 fibroblasts. As shown in FIGS. 2, 3 and 4 there was a significant difference in sensitivity of eukaryotic cells to PlcHR₂. HUVECs and CHO cells were extremely sensitive to PlcHR₂, requiring only picomolar concentrations to induce LDH release, whereas HeLa, L929 fibroblasts, and primary lung epithelial cells were resistant to PlcHR₂ up to 4 μg/ml PlcHR₂. See FIGS. 2, 3 and 4. In FIG. 4, cells were treated with increasing concentration of PlcHR₂ for 6 hours at which time cytotoxicity was measured by LDH release.

### PlcHR₂ Induces Activation of Caspase-3 in HUVEC

As stated above PlcHR₂ is cytotoxic to HUVEC. It is believed that aspartate-specific cysteinyl proteases or caspases are important proteases in the apoptotic process. One of these caspases, caspase-3, is believed to be a key mediator of apoptosis in mammalian cells and its activation is believed to be an indication of apoptosis.

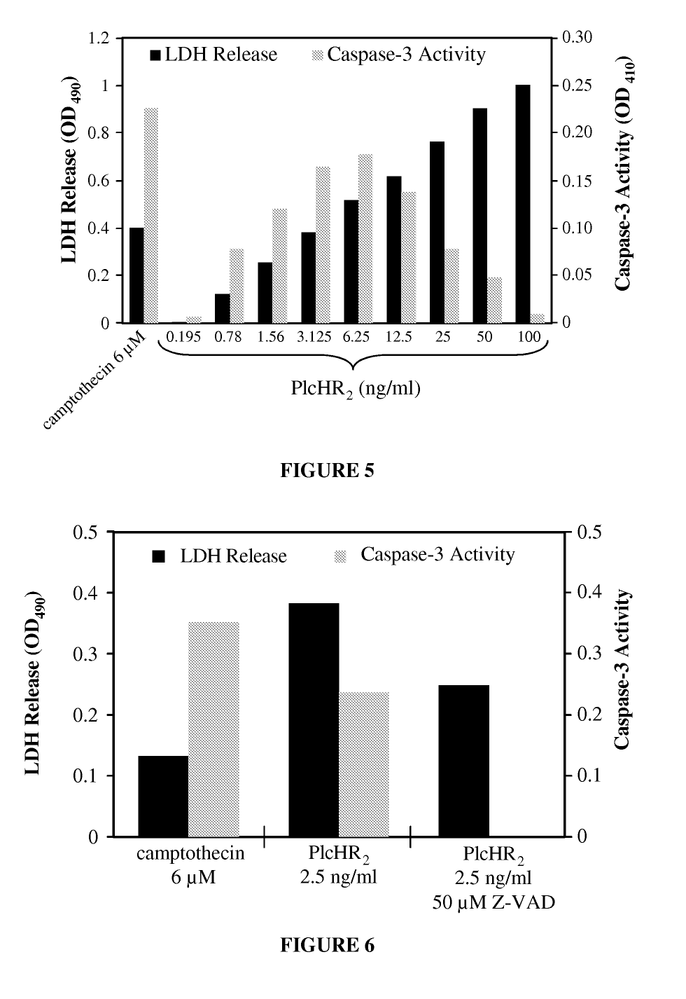

Treatment of HUVEC with picomolar concentration of PlcHR₂ resulted in activation of caspase-3 (see FIG. 5). Both caspase-3 activation and LDH release were measured at 16 hours post treatment with increasing concentrations of PlcHR₂. As shown in FIG. 5, caspase-3 activity increased as the concentration of PlcHR₂ increased until it peaked at 6.25 ng/ml PlcHR₂. The level of caspase-3 activity induced with 6.25 ng/ml PlcHR₂ was similar to cells treated with the apoptotic control camptothecin (6 μM). Beyond 6.25 ng/ml PlcHR₂, caspase-3 activity begun to decrease but the release of LDH increased until approximate at 100 ng/ml PlcHR₂ there was very little caspase-3 activity and LDH release had substantially reached its maximum. The addition of the pan-caspase inhibitor Z-VAD-FMK inhibited substantially all PlcHR₂ activation of caspase-3 and reduced the amount of LDH release. Without being bound by any theory, it is believed that this was an indication that at least a portion of the cells releasing LDH were dying by apoptosis (see FIG. 6). This data appears to suggest that at lower concentration of PlcHR₂ (<12.5 ng/ml) the cells were both necrotic and apoptotic but at greater concentrations of PlcHR₂ the majority of the cells were necrotic.

PlcHR₂ and the Clostridium perfringens α-toxin Inhibit Endothelial Cell Migration
Migration Assays Were Conducted using HUVECs in the BD BioCoat

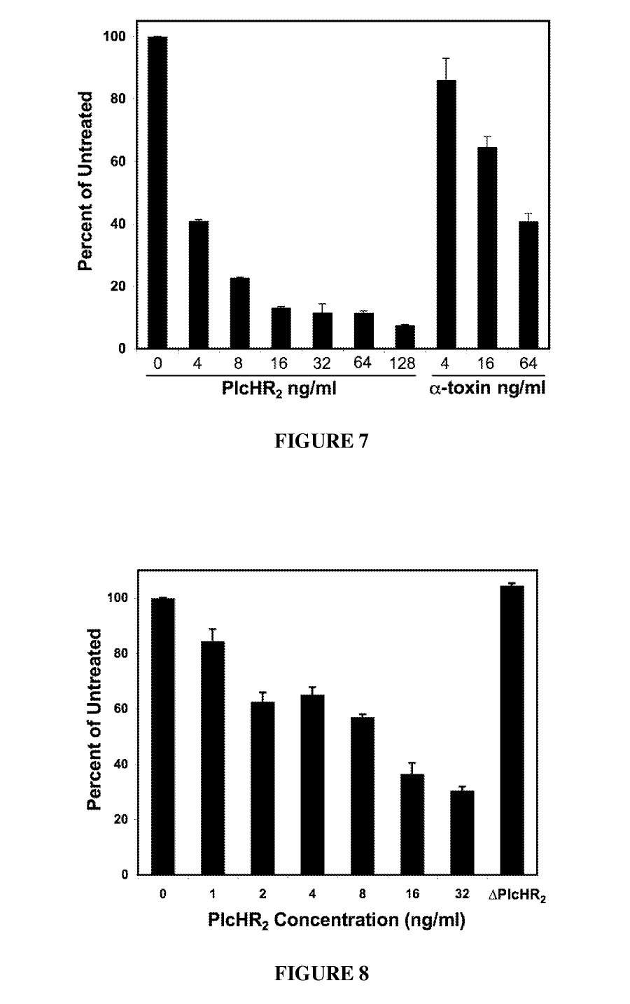

Migration Angiogenesis System using 2% fetal bovine serum and 10 ng/ml VEGF as chemoattractants. The data showed that PlcHR₂ and the Clostridium perfringens α-toxin inhibited endothelial migration in a dose dependent manner. See FIG. 7. The concentration that resulted in 50% inhibition of migration (IC₅₀) for PlcHR₂ and α-toxin were calculated to be 3.25 ng/ml and 45 ng/ml, respectively.

### PlcHR₂ Inhibits Endothelial Cell Invasion

Endothelial cell invasion assays were conducted using human umbilical vein endothelial cells (HUVECs) in the BD BioCoat Invasion Angiogenesis System (BD Biosciences, Bedford, Mass.) using 2% fetal bovine serum and 10 ng/ml vascular endothelial growth factor (VEGF) as chemoattractants. The data showed that PlcHR₂ inhibited endothelial cell invasion in a dose dependent manner. See FIG. 8. As shown in FIG. 8, heat inactivated PlcHR₂ (i.e., ΔPlcHR₂) did not significantly inhibit endothelial cell invasion. It is believed that this indicates that the observed phenotype was due to the activity associated with PlcHR₂ and not heat stable Lipopolysaccharide (LPS). See FIG. 8, ΔPlcHR₂. The IC₅₀ for PlcHR₂ in this assay was calculated to be about 10 ng/ml.

### PlcHR₂ Inhibits Endothelial Cell Proliferation

PlcHR₂ was tested for its ability to suppress capillary endothelial cell proliferation in vitro and found that PlcHR₂ inhibited both basal and mitogen driven endothelial cell proliferation. See Table 2. As shown in Table 2, PlcHR₂ appeared to inhibit the mitogen stimulated endothelial cells better than the unstimulated.

TABLE 2
PlcHR₂ inhibits both basal and mitogen
driven endothelial cell proliferation.

| | | Mitogen Stimulated |
|---|---|---|
| PlcHR₂ Concentration | Basal | (1 ng/ml bFGF) |
| 0.01 ng/ml | 0% | 0% |
| 0.10 ng/ml | 0% | 0% |
| 1.0 ng/ml | 0% | 0% |
| 10 ng/ml | 33% | 75% |
| 100 ng/ml | 62% | 100% |

bFGF is basic fibroblast growth factor.

### PlcHR₂ Inhibits Endothelial Cell Tube Formation

One of the tests for angiogenesis is the measurement of the ability of endothelial cells to form capillary like structures (Tubes) when grown in vitro on Matrigel™. The tube formation assay allows assessment of attachment, invasion, migration, and differentiation into capillary-like structures as well as the modulation of these events by inhibitory compounds.

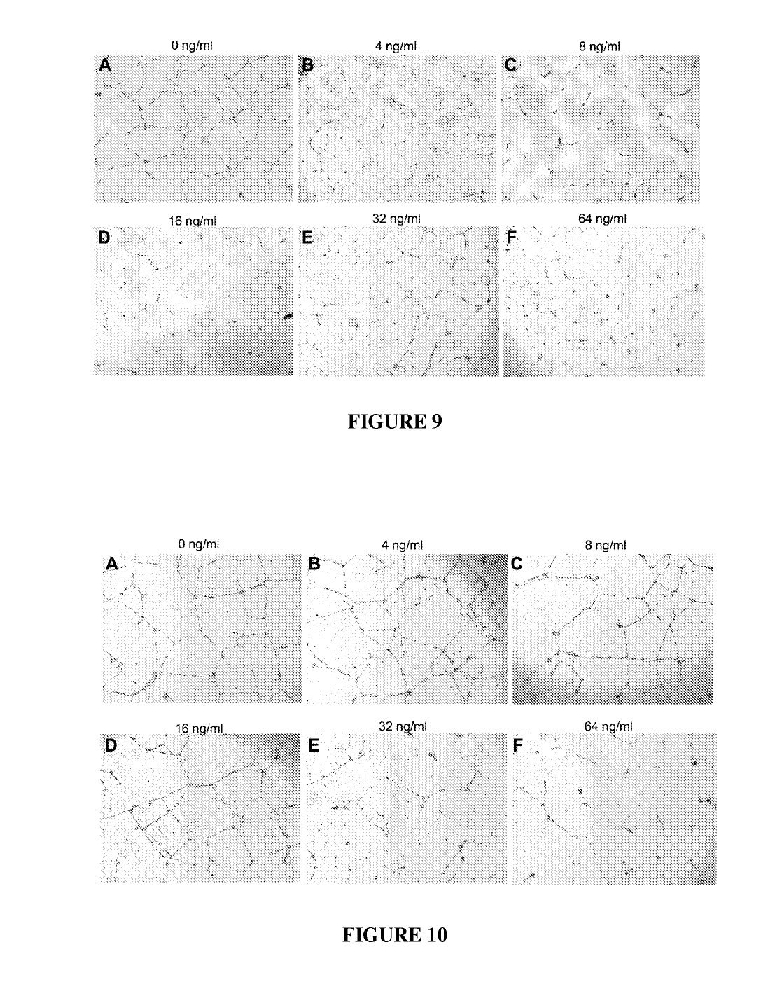

As shown in FIG. 9, 4 ng/ml of PlcHR₂ inhibited endothelial cell tube formation on matrigel. Endothelial cell were grown on matrigel and challenged with 4-64 ng/ml PlcHR₂ for 20 hours at which time tube formation was visualized (20X). It should be noted that when the media containing PlcHR₂ was exchanged with fresh media after the initial 20 hour incubation, tubes formed within 24 hours indicating that PlcHR₂ was not necessarily killing the endothelial cells but was inhibiting the tube formation process (data not shown). It is believed that the quiescent endothelial cells of the vasculature lie in a protective state in that they express genes that prevent up-regulation of pro-inflammatory genes and anti-apoptotic genes. This protective state is believed to be important in terms of treatment in that anti-angiogenesis drug should not target or should be less selective towards the already established vasculature of the body.

This protective state was studied by allowing the endothelial cells to form tubes for 24 hours prior to challenge with PlcHR₂. As shown in FIG. 10, increased concentrations of PlcHR₂ are required to break down endothelial tubes after they have already formed. In FIG. 10, endothelial cells were grown on matrigel for 24 hours at which time they were challenged with 4-64 ng/ml of PlcHR₂ for 20 hours. Tube formation was visualized (20X) and photographed. FIG. 10 shows that when tubes were already formed it took about 32 ng/ml of PlcHR₂ to break down the tubes compared to 4 ng/ml of PlcHR₂ to prevent tube formation when the tubes have not yet formed (see FIG. 9). This result indicates that PlcHR₂ was preferentially targeting activated endothelial cells, which is desired target for an anti-angiogenesis activity.

Inhibition of Angiogenesis in vivo by PlcHR₂

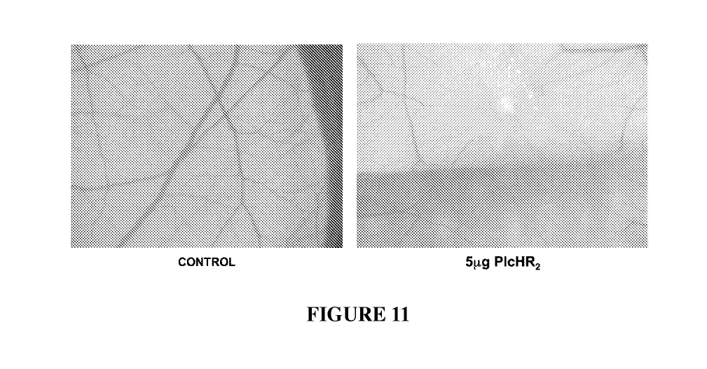

Effectiveness of PlcHR₂ at inhibiting endothelial cell migration, invasion, proliferation, and tube formation have been shown as described above. PlcHR₂ was also tested as an inhibitor of angiogenesis in the in vivo chicken chorioallantonic membrane (CAM) assay. As shown in FIG. 11, the large avascular zone caused by 5 ng of PlcHR₂ showed significant inhibition of embryonic neovascularization.

PlcHR₂'s Phospholipase C Activity is Required for it's Anti-Angiogenesis Activity

Using a random mutagenic protocol a PlcH mutant (Thr178A1a) deficient in enzymatic activity was isolated. The T178A mutant is about 30 times less enzymatically active as wild type PlcHR₂ (see Table 3). Recently, the structure of the Francisella tularensis AcpA, a PlcH homolog, was solved and Serine 175 found in the enzymes active site was identified as essential for catalysis of substrates. See Felts et al., J Biol Chem, 2006, 281(40), 30289-30298. An AcpA Ser175Ala mutant exhibited no detectable enzymatic activity. Id. When the F. tularensis AcpA was aligned with PlcH the AcpA Serine 175 corresponded to Threonine 178 in PlcH suggesting that the T178A PlcH mutant is also an active site mutant. The T178A PlcH mutant was greatly reduced in its ability to inhibit endothelial cell invasion (Table 3) indicating that the PLC activity of PlcH is required for its anti-angiogenic properties.

TABLE 3
PlcHR₂ phospholipase C activity is required
for it's anti-angiogenesis activity.

| | Vₘₐₓ | Kₘ | k_{cat} | IC₅₀ Endothelial |
|---|---|---|---|---|
| Sample | (μmol · min⁻¹ · mg⁻¹) | (μM) | (s⁻¹) | Invasion Assay |
| PlcHR₂ | 147 ± 6 | 19 ± 2.9 | 192 | 10 ng/ml |
| T178A | 4.85 ± 1 | 200 ± 57 | 6 | 1200 ng/ml |
| Mutant | | | | |

The initial rates of hydrolysis (μmol · min⁻¹ · mg⁻¹) for the NPPC assays were fitted to the Michaelis-Menten equation using the program Sigma Plot (SPSS Inc.). The kinetic parameters Vₘₐₓ and Kₘ were obtained from the nonlinear least squares fit of the data; k_{cat} values were calculated using a Mᵣ of 96,000 Da for PlcHR₂. Each set of data is from four independent determinations. Endothelial cell invasion was measured with the BD angiogenesis invasion kit.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1. A method for treating a disease or condition associated with angiogenesis in a subject, said method comprising administering a phospholipase C to the subject such that the phospholipase C reduces angiogenesis activity in said subject.

2. The method of Claim Error! Reference source not found., wherein the phospholipase C binds to an integrin receptor.

3. The method of Claim Error! Reference source not found., wherein the disease or condition associated with angiogenesis is cancer, macular degeneration, arthritis, or an infectious disease.

4. The method of Claim Error! Reference source not found., wherein the phospholipase C is a bacterial extracellular phospholipase C.

5. The method of Claim Error! Reference source not found., wherein the phospholipase C is selected from the group consisting of PlcHR₂, Clostridium perfringens α-toxin, and a mixture thereof.

6-15. (canceled)

16. A method for inhibiting abnormal fibrovascular growth in a mammal comprising administering to a mammal having abnormal fibrovascular growth a phospholipase C in an amount effective to inhibit abnormal fibrovascular growth in the mammal.

17. The method of Claim Error! Reference source not found., wherein the phospholipase C binds to an integrin receptor and inhibits abnormal fibrovascular growth in the mammal.

18. The method of Claim Error! Reference source not found., wherein the abnormal fibrovascular growth is associated with inflammatory arthritis.

19. A method of inhibiting a proliferative retinopathy in a mammal comprising administering to a mammal having proliferative retinopathy a phospholipase C in an amount effective to reduce the proliferative retinopathy in the mammal.

20. The method of Claim Error! Reference source not found., wherein the proliferative retinopathy occurs as a result of diabetes or aging in the mammal.

21. (canceled)
